(19) United States
(12) Patent Application Publication
(10) Pub. No.: US 2012/0295286 A1
(43) Pub. Date: Nov. 22, 2012

(54) Methods for the Diagnosis, Prognosis and Monitoring of Cancer Therapy Using BP1

(75) Inventor: Patricia E. Berg, Washington, DC (US)

(73) Assignee: The George Washington University, Washington, DC (US)

(21) Appl. No.: 13/453,432

(22) Filed: Apr. 23, 2012

Related U.S. Application Data
(60) Provisional application No. 61/477,963, filed on Apr. 21, 2011.

(51) Int. Cl. G01N 33/574; G01N 21/64

(52) U.S. Cl. 435/7.92; 436/501

(57) ABSTRACT

The invention described herein relates to method for diagnosing or monitoring the progression of a cancer, e.g., breast cancer, prostate cancer or brain cancer, in a subject by determining the amount of one or more biomarkers in a bodily fluid sample, where the biomarkers comprise pBP1. The invention described herein also relates to method for assessing the efficacy of a treatment of a subject having or suspected of having a cancer, by determining the amount of one or more biomarkers in a bodily fluid sample.

### FIELD OF THE INVENTION

The present invention relates to the fields of medical diagnosis, patient monitoring, and treatment efficacy evaluation.

### BACKGROUND OF THE INVENTION

Beta protein 1 (“BP1”) is a transcription factor belonging to the homeobox gene family (Chase, et al. “BP1, a homeodomain-containing isoform of DLX4, represses the beta-globin gene”, Mol Cell Biol 22 (2002), 2505-2514; see also U.S. Pat. No. 6,416,956 incorporated herein by reference). It has previously been reported that BP1 is activated in 80% of women with breast cancer and is associated with clinically aggressive breast cancer and there is a statistical association between BP1 positivity and ER negativity, large tumor size, increased cell proliferation, and metastasis. BP1 is also linked to progression of breast cancer: the percentage of BP1 positive cases increased from 0% in normal tissue to 21% in ductal hyperplasia, 46% in ductal carcinoma in situ, and 80% in invasive ductal carcinoma (Fu et al. “Correlation of expression of BP1, a homeobox gene, with estrogen receptor status in breast cancer”, Breast Cancer Res 5:82-87 (2003) and Man et al., “Expression of BP1, a homeobox gene, correlates with progression and invasion of mammary ductal carcinoma”, Breast Cancer Res Treat 90:241-247 (2005)). Prior to the invention described herein, BP1 had not been reported to be secreted and was not reported to be secreted in exosomes.

Exosomes are membrane bound nanoparticles that are formed through inward budding of endosomal membranes giving rise to intracellular multivesicular bodies that later fuse with the plasma membrane, releasing the exosomes to the exterior (Thery et al., “Exosomes: compositions, biogenesis and function” Nat. Rev. Immunol 2:569-79 (2002)). It has also been reported that there is a more direct release of exosomes. Certain cells, such as Jurkat T-cells, are said to shed exosomes directly by outward budding of the plasma membrane (Booth et al., “Exosomes and HIV Gag bud from endosome-like domains of the T cell plasma membrane” J. Cell biol. 172:923-35 (2006)). Exosomes have been reported to be shed from many different cell types, normal, diseased and neoplastic (Thery et al., “Exosomes: composition, biogenesis and function” Nat Rev Immunol. 2:569-79 (2002)) and were first described as a mechanism to discard transferrin-receptors from the cell surface of maturing reticulocytes (Pan and Johnstone, “Fate of the transferrin receptor during maturation of sheep reticulocytes in vitro: selective externalization of the receptor” Cell 33:967-78 (1983)).

Exosomes have diameters of approximately 30-100 nm and are present in blood and other bodily fluids. They contain a variety of molecules including signal peptides, mRNA, microRNA, and lipids. Exosomes can function to export unneeded endogenous molecules and therapeutic drugs from cells. When exosomes are taken up by specific cells, they may act locally to provide autocrine or paracrine signals or, at a distance, as a newly described nanoparticle-based endocrine system. Specifically, mRNA transferred to cells by exosomes can result in the production of new proteins. In cancer, signals via exosomes affect the immune system by inhibiting the functions of T cells and normal killer (NK) cells and by inhibiting the differentiation of precursors to mature antigen-presenting cells. Also, exosomes increase the number and/or activity of immune suppressor cells, including myeloid-derived suppressor cells, T-regulatory cells, and CD14⁺, HLA-DR^{-/low} cells (see Zhang and Grizzle, “Exosomes and Cancer: A Newly Described Pathway of Immune Suppression” Clin Cancer Res 17:959-964 (Mar. 1, 2011; Published OnlineFirst Jan. 11, 2011).

Exosomes derived from B-cells and dendritic cells are reported to have potent immuno-stimulatory and antitumor effects in vivo and have been used as antitumor vaccines (Chaput et al., “The Potential of exosomes in immunotherapy” Expert Opin Biol Ther. 5:737-47 (2005)). Dendritic cell-derived exosomes are reported to contain the co-stimulatory proteins necessary for T-cell activation, whereas most tumor cell-derived exosomes do not (Wieckowski and Whiteside, “Human tumor-derived and dendritic cell-derived exosomes have distinct biologic roles and molecular profiles” Immuol Res. 36:257-54 (2006)).

Exosomes isolated from tumor cells may act to suppress the immune response and accelerate tumor growth (Clayton et al., “Human tumor-derived exosomes selectively impair lymphocyte responses to interleukin-2” Cancer Res 67:7458-66 (2007); Liu et al., “Murine mammary carcinoma exosomes promote tumor growth by suppression of NK cell function” J. Immunol. 176:1375-85 (2006)). Some have suggested that breast cancer exosomes may stimulate angiogenesis, and that platelet-derived exosomes may promote tumor progression and metastasis of lung cancer cells (Janowska-Wieczorek et al., “Exosomes derived from activated platelets induce metastasis and angiogenesis in lung cancer” Int J Cancer 113:752-60 (2005); Millimaggi et al., “Tumor vesicle associated CD147 modulates the angiogenic capability of endothelial cells” Neoplasia 9:349-57 (2007)).

### SUMMARY OF THE INVENTION

We have discovered that the transcription factor, BP1 protein (“pBP1”), is secreted and can be found in bodily fluids, including, e.g., blood and serum, in particular in exosomes that are present in bodily fluids. Until this discovery the secretion of transcription factors in exosomes was unknown. Furthermore, it likely that pBP1 will be detected at higher levels in bodily fluids, particularly in the exosomes, from women with breast cancer, particularly hyperplasia (“IDH”), ductal carcinoma in situ (“DCIS”), invasive ductal carcinoma (“IDC”), or inflammatory breast cancer (“IBC”), as compared to a control bodily fluids, e.g., the serum from patients without breast cancer (“NHS”).

In general, the invention described herein is a method for detecting in a subject the presence or absence of a one or more of biomarkers that are associated with a cancer thereby aiding the diagnosis, monitoring and evaluation of a cancer, e.g., detecting pBP1 in exosomes as an indicator of a breast cancer, e.g., any of IDH, DCIS, IDC, and IBC. The invention described herein is also a method using the exosome biomarkers for evaluating a treatment efficacy. A biomarker may be a nucleic acid molecule, a peptide or polypeptide which is associated with a cancer. For example, the biomarker is a peptide or polypeptide wherein the presence of the biomarker or an increase in the amount of the biomarker is associated with the presence or progression of a cancer. Preferably the biomarker is a peptide or a polypeptide wherein the presence of the biomarker or an increase in the amount of the biomarker is associated with the presence or progression of a cancer, e.g., pBP1.

One aspect of the invention is a method for aiding in the diagnosis or monitoring of a cancer in a subject, comprising the steps of: a) isolating an exosome-containing bodily fluid sample from the subject; and b) detecting the presence or absence of one or more biomarkers within the sample, wherein the biomarker is associated with a cancer. The presence of pBP1 has been shown to be associated with prostate cancer and also with breast cancer. In particular increased levels of pBP1 as compared to normal prostate or breast tissue has been associated with prostate cancer and breast cancer, e.g., IDH, DCIS, IDC and IBC respectively. Thus in an embodiment of this invention the biomarker may comprise a pBP1 and the cancer may be breast cancer. The methods may further comprise the step or steps of comparing the result of the detection step to a control (e.g., comparing the amount of one or more biomarkers detected in the sample to one or more control levels), wherein the subject is diagnosed as having the cancer if there is a measurable difference in the result of the detection step as compared to a control.

Another aspect of the invention is a method for aiding in the evaluation of treatment efficacy in a subject, comprising the steps of: a) isolating a exosome-containing bodily fluid sample from the subject; and b) detecting the presence or absence of one or more biomarker, e.g., pBP1, within the sample, wherein the biomarker is associated with the treatment efficacy for a cancer. The method may further comprise the step of providing a series of a bodily fluid samples over a period of time from the subject. Additionally, the method may further comprise the step or steps of determining any measurable change in the results of the detection step (e.g., the amount of one or more detected biomarkers) in each of the bodily fluid samples from the series to thereby evaluate treatment efficacy for the cancer.

In certain preferred embodiments of the foregoing aspects of the invention, the bodily fluid sample, e.g., milk, tears, urine, saliva, sputum, blood, serum, plasma, ascites, cyst fluid, pleural fluid, cerebral spinal fluid, or combinations thereof. Particularly preferred bodily fluids are blood and serum.

In some embodiments of the foregoing aspects of the invention, the methods further comprise the isolation of a selective exosome-containing fraction derived from cells of a specific type (e.g., cancer or tumor cells) or bodily fluid. Additionally, the selective exosome fraction may consist essentially of exosomes obtained from serum.

In embodiments of the foregoing aspects of the invention, the biomarker associated with a cancer is a pBP1 or a pBP1 and a vesicular endothelial growth factor, VEGF. The biomarkers may also comprise, e.g., hMAM (mamaglobin), Her2, EGFR, CEA, CD63, or BRCA1 (breast cancer type 1 susceptibility protein).

In embodiments of the foregoing aspects of the invention, the cancer is a breast cancer a prostate cancer, a colorectal cancer, a ovarian, a leukemia, a lung cancer, or a brain cancer.

Another aspect of the invention is a method for aiding in the diagnosis or monitoring to progression of a cancer in a subject, comprising the steps of a) obtaining a bodily sample from the subject; b) obtaining exosomes from the sample and c) assaying the exosome for the presence of one or more biomarkers associated with a cancer, wherein the concentration of the one or more biomarkers aids in the diagnosis or monitoring of a cancers. In methods where multiple samples are obtained from the subject a change in the concentration of the biomarkers in the exosome samples over time aids in the diagnosis and progression of the condition.

Various aspects and embodiments of the invention will now be described in detail. It will be appreciated that modification of the details may be made without departing from the scope of the invention. Further, unless otherwise required by context, singular terms shall include pluralities and plural terms shall include the singular.

### BRIEF DESCRIPTION OF THE DRAWINGS

FIG. 1A depicts a Western blot of pBP1 in exosomes of MDA-MB-231 cells. Lane 1, conditioned media before concentration (very low amount of exosomal protein expected); lane 2, supernatant after centrifugation at 100,000 g; lane 3, 1×PBS after washing of the exosomal pellet; lane 4, exosomes (Exo); lane 5, cellular extract (Cell Ex). The upper panel designates the expected larger, approximately 37 kDa, pBP1 band, referred to hereinafter as pBP-1³⁷. The bands in the lower panel demonstrate the presence of actin in both the exosomal fraction as well as in the cellular extract.

FIG. 1B is an electron microscopy of exosomes from MCF7/O2 cells. Arrows indicate exosomes, which exhibit the characteristic cup-shaped morphology (Thery et al. “Isolation and characterization of exosomes from cell culture supernatants and biological fluids” Curr. Protoc. Cell Biol. Chapter 3: Unit 3.22 (2006)). The two smaller vesicles are nonexosomal vesicles isolated with the exosomes during ultracentrifugation, described by Thery et al, 2006.

FIG. 2 depicts a Western Blot of pBP1 detected in exosomes and supernatants from BCS (A) and NHS (B).

FIG. 3. Competition for pBP1³⁷ band. The antibody was pre-incubated with 10 μg of the peptide, 5× more than the amount of the anti-BP1 antibody, prior to Western blot analysis. Cell extracts from 01B cells (MCF7 derivative engineered to overexpress pBP1) were in duplicate.

FIG. 4. Effect of exosomes derived from cells overexpressing pBP1 on cell growth. Cells were grown in serum free DMEM as bovine serum contains exosomes

### DETAILED DESCRIPTION OF THE INVENTION

Exosomes are small microvesicles approximately 30-100 nm that are released by exocytosis of intracellular multivesicular bodies. Certain aspects of the present invention are based on the surprising finding that pBP1 is secreted in exosomes and can be isolated from the serum and are expected to be isolated from other bodily fluids of breast cancer patients. This is the first discovery of pBP1 containing exosomes present in a bodily fluid of a subject. Prior to this discovery it was not known that transcription factors such as BP1 were secreted in exosomes. The pBP1 found within the exosomes described herein, as well as other contents of the exosomes such as, mRNA, microRNAs, DNAs and other polypeptides that are associated with cancer, can be used as valuable biomarkers for tumor diagnosis, characterization and prognosis by providing a genetic profile. Contents within these exosomes can also be used to monitor tumor progression over time by analyzing concentration of pBP1 in the exosomes, or the number of pBP1-containing exosomes in a bodily fluid, during tumor progression for an increase or decrease in concentration over time, or over a course of treatment. In some embodiments the presence or concentrations other biomarkers in addition to pBP1 can also be used for tumor diagnosis, characterization and prognosis.

The concentration of pBP1 in a subject's bodily fluid may increase as the tumor grows in which case the concentration of the pBP1 in bodily fluids is proportional to the corresponding tumor load. It is expected that the greater the tumor load, the higher the concentration of pBP1 in bodily fluids.

One aspect of the present invention relates to methods for detecting, diagnosing, monitoring, treating or evaluating a cancer in a subject by determining the concentration of pBP1 in exosomes in a bodily fluid. The determination may be performed using the bodily fluid sample without first isolating the exosomes. Alternatively the determination may be performed using by first isolating the exosomes from a bodily fluid sample.

Another aspect of the present invention relates to methods for detecting, diagnosing, monitoring, treating or evaluating a cancer in a subject comprising the steps of, isolating exosomes from a bodily fluid of a subject, and analyzing the exosomes for one or more biomarker(s), wherein the biomarker includes, e.g., a polypeptide associated with a cancer, e.g., pBP1. The biomarkers are analyzed qualitatively and/or quantitatively, and the results are compared to results expected or obtained for one or more other subjects who have or do not have the cancer. For example, the presence of a difference in exosome polypeptide content of the subject, as compared to that of one or more other individuals, is indicative of the presence or absence of, or the progression of (e.g., changes of tumor size and/or tumor malignancy), or the susceptibility of a subject to a cancer. Preferably the polypeptides comprise pBP1 and the amount of pBP1 is analyzed.

The exosomes are preferably isolated from a sample taken of a bodily fluid from a subject. As used herein, a “bodily fluid” refers to a sample of fluid isolated from anywhere in the body of the subject, preferably a peripheral location, including but not limited to, for example, blood, plasma, serum, urine, sputum, saliva, spinal fluid, cerebrospinal fluid, pleural fluid, breast cyst aspirates, breast milk, fluid of the respiratory, intestinal, and genitourinary tracts, tear fluid, fluid from the lymphatic system, lymph fluid, semen, intra-organ system fluid, ascitic fluid, tumor cyst fluid, amniotic fluid and combinations thereof.

The term “subject” is intended to include all animals shown to or expected to have exosomes. In particular embodiments, the subject is a mammal, a human or nonhuman primate, a dog, a cat, a horse, a cow, other farm animals, or a rodent (e.g. mice, rats, guinea pig. etc.). The term “subject” and “individual” are used interchangeably herein. A “subject in need thereof” is a subject who has, or is suspected of having, a cancer.

Methods of isolating exosomes from a biological sample, including bodily fluids, are known in the art. For example, a method of differential centrifugation is described in a paper by Raposo et al. (Raposo et al., B lymphocytes secrete antigen-presenting vesicles. J Exp Med. 183:1161-721996), and similar methods are detailed in the Examples presented herein.

Methods of anion exchange and/or gel permeation chromatography are described in U.S. Pat. Nos. 6,899,863 and 6,812,023. Methods of sucrose density gradients or Organelle electrophoresis are described in U.S. Pat. No. 7,198,923. A method of magnetic activated cell sorting (MACS) is described in (Taylor and Gercel-Taylor, “MicroRNA signatures of tumor-derived exosomes as diagnostic biomarkers of ovarian cancer” Gynecol Oncol. 110:13-21 (2008)). A method of nanomembrane ultrafiltration concentrator is described in (Cheruvanky et al., “Rapid isolation of urinary exosomal biomarkers using a nanomembrane ultrafiltration concentrator” Am J Physiol Renal Physiol. 292:F1657-61 2007). Preferably, exosomes can be identified and isolated from bodily fluid of a subject by a microchip technology that uses a unique microfluidic platform to efficiently and selectively separate tumor derived exosomes. This technology, as described in a paper by Nagrath et al. (Nagrath et al., “Isolation of rare circulating tumour cells in cancer patients by microchip technology” Nature. 450:1235-9 (2007)), can be adapted to identify and separate exosomes using similar principles of capture and separation as taught in Nagrath et al. 2007. Each of the foregoing references is incorporated by reference herein for its teaching of these methods.

In an embodiment of this invention, the exosomes isolated from a bodily fluid are enriched for those originating from a specific cell type, for example, breast, prostate, brain, lung, pancreas, stomach, intestine, bladder, kidney, ovary, testis, skin, colorectal, esophagus, liver, placenta, fetus cells. Because the exosomes often carry surface molecules such as antigens from their donor cells, surface molecules may be used to identify, isolate and/or enrich for exosomes from a specific donor cell type (Al-Nedawi et al., “Intercellular transfer of the oncogenic receptor EGFRvIII by microvesicles derived from tumour cells” Nat Cell Biol. 10:619-24 (2008); Taylor and Gercel-Taylor, 2008). In this way, exosomes originating from distinct cell populations can be analyzed for their nucleic acid and polypeptide content. For example, tumor (malignant and non-malignant) exosomes carry tumor-associated surface antigens and may be detected, isolated and/or enriched via these specific tumor-associated surface antigens. In one example, the surface antigen is epithelial-cell-adhesion-molecule (EpCAM), which is specific to exosomes from carcinomas of lung, colorectal, breast, prostate, head and neck, and hepatic origin, but not of hematological cell origin (Balzar et al., “The biology of the 17-1A antigen (Ep-CAM)” J Mol Med. 77:699-712 (1999); Went et al., “Frequent EpCam protein expression in human carcinomas” Hum Pathol. 35:122-8 (2004)). In another example, the surface antigen is CD24, which is a glycoprotein specific to urine exosomes (Keller et al., “CD24 is a marker of exosomes secreted into urine and amniotic fluid” Kidney Int. 72:1095-102 (2007)). In yet another example, the surface antigen is selected from a group of molecules CD70, carcinoembryonic antigen (CEA), EGFR, EGFRvIII and other variants, Fas ligand, TRAIL, tranferrin receptor, p38.5, p97, HSP72, CD63, and HER2. Additionally, tumor specific exosomes may be characterized by the lack of surface markers, such as CD80 and CD86.

The isolation of exosomes from specific cell types can be accomplished, for example, by using antibodies, aptamers, aptamer analogs or molecularly imprinted polymers specific for a desired surface antigen. In one embodiment, the surface antigen is specific for a cancer type. In another embodiment, the surface antigen is specific for a cell type which is not necessarily cancerous. One example of a method of exosome separation based on cell surface antigen is provided in U.S. Pat. No. 7,198,923. As described in, e.g., U.S. Pat. Nos. 5,840,867 and 5,582,981, WO12003/050290 and a publication by Johnson et al. (Johnson et al., “Surface-immobilized peptide aptamers as probe molecules for protein detection” Anal Chem. 80:978-83 (2008)), aptamers and their analogs specifically bind surface molecules and can be used as a separation tool for retrieving cell type-specific exosomes. Molecularly imprinted polymers also specifically recognize surface molecules as described in, e.g., U.S. Pat. Nos. 6,525,154, 7,332,553 and 7,384,589 and a publication by Bossi et al. (Bossi et al., “Exosome-mediated transfer of mRNAs and microRNAs is a novel mechanism of genetic exchange between cells” Nat Cell Biol. 9:654-9 (2007)) and are a tool for retrieving and isolating cell type-specific exosomes. Each of the foregoing reference is incorporated herein for its teaching of these methods.

The analysis of pBP1 present in the exosomes may be quantitative and/or qualitative. For quantitative analysis, the amounts (expression levels), either relative or absolute, of the biomarkers within the exosomes are measured with methods known in the art (described below). Exosomal polypeptides may be released from exosomes by any method known in the art, e.g., SDS, freeze-thawing, sonication or Na₂CO₃ (carbonate) treatment. For qualitative analysis, the content of exosomes may be analyzed, e.g., by Southern, Northern, PCR or real time PCR or Western blot analysis using nucleic acid primers or antibodies that are specific for a particular biomarker, e.g., antibodies that specifically bind to one or more of pBP1, pBP1³⁷ or pBP1²⁵. Antibodies which bind pBP1 are known in the art, e.g., U.S. Pat. No. 6,416,956 describes a polyclonal antibody raised in rabbits against the unique N-terminal BP1 peptide SYPYTEPANPGSYLSCQQ (SEQ ID NO:1). Methods for generating polyclonal and monoclonal antibodies suitable for use in ELISA and Western blot analysis are well known in the art.

In one embodiment, the analysis is of a profile of the amounts (levels) of the one or more biomarkers present in the exosome, herein referred to as a “quantitative biomarker profile” of the exosomes. In another embodiment, the analysis is of a profile of the species of specific nucleic acids present in the exosomes (both wild type as well as variants), herein referred to as a “nucleic acid species profile.” In another embodiment, the analysis is of a profile of the polypeptides present in the exosomes (both wild type as well as variants), herein referred to as a “polypeptide species profile.” A term used herein to refer to a combination of these types of profiles is “genetic profile” which refers to the determination of the presence or absence of nucleotide species, nucleotide or polypeptide variants and also increases or decreases in nucleic acid levels. For example, it has been reported that BP1 is amplified in some cancers, e.g., breast cancers. Cavalli et al., Cancer Genetics and Cytogenetics 1(87)19-24 (2008).

Once generated, these genetic profiles of the exosomes are compared to those expected in, or otherwise derived from a healthy normal individual. A profile can be a genome wide profile (set up to detect all possible expressed genes, DNA sequences or polypeptides). It can be narrower as well, such as a cancer wide profile (set up to detect all possible genes or nucleic acids derived therefrom, or polypeptides known to be associated with one or more cancers). Where one specific cancer is suspected or known to exist, the profile can be specific to that cancer (e.g., set up to detect all possible genes or nucleic acids derived therefrom, or polypeptides associated with various clinically distinct subtypes of that cancer or known drug-resistant or sensitive mutations of that cancer).

The entire polypeptide or nucleic acid molecule content of the exosomes or only a subset of specific polypeptides or nucleic acid molecules that are likely or suspected of being influenced by the presence of a cancer, e.g., breast cancer, prostate cancer or brain cancer, can be amplified and/or analyzed. The identification of a polypeptide aberration(s) in the analyzed exosome can be used to diagnose the subject for the presence of a condition, e.g., breast cancer, with which that aberration(s) is associated. For instance, analysis for the presence or absence of pBP1, or pBP1 and another biomarker associated with a cancer, e.g. VEGF, hMAM (mamaglobin), Her2, EGFR, CEA, or BRCA1 can indicate the cancer's presence in the individual. Alternatively, or in addition, analysis of polypeptides for an increase or decrease in polypeptide levels specific to a cancer, e.g., breast cancer, prostate cancer or brain cancer, can indicate the presence of the cancer in the individual (e.g., a relative increase in pBP1 from exosomes).

Determining the presence or absence of one or more biomarkers in an exosome can be performed in a variety of ways known in the art. A variety of methods are available for such analysis, including, but not limited to, Western blotting, silver staining, mass spectrometry, PCR, and real time PCR.

Because we have discovered that pPB1 is secreted in exosomes, the presence and/or amount of pBP1 in exosomes reflects the presence of tumor cells, as demonstrated herein. Furthermore, methods of diagnosis using exosomes have advantages over other methods of diagnosis performed directly on a tumor biopsy sample. For example, obtaining a bodily fluid from a subject for assay can be less invasive than obtaining a biopsy sample from a subject, and can be useful in detecting tumor cells that are present in numbers too small to be readily detectable by other means, e.g., x-ray, mammogram, sonogram or biopsy. Another advantage of the analysis of exosome polypeptides, as opposed to other forms of sampling of tumor/cancer polypeptides, is the availability for analysis of tumor/cancer polypeptides derived from all foci of a tumor or genetically heterogeneous tumors present in an individual. Biopsy samples are limited in that they provide information only about the specific focus of the tumor from which the biopsy is obtained. Different tumorous/cancerous foci found within the body, or even within a single tumor often have different genetic profiles and are not analyzed in a standard biopsy. However, analysis of the exosome nucleic acids or polypeptides from an individual presumably provides a sampling of all foci within an individual. This provides valuable information with respect to recommended treatments, treatment effectiveness, disease prognosis, and analysis of disease recurrence, which cannot be provided by a simple biopsy.

Also described herein are methods for monitoring disease (e.g. cancer) progression in a subject, and also to a method for monitoring disease recurrence in an individual. These methods comprise the steps of isolating exosomes from a bodily fluid of an individual, as discussed herein, and analyzing one or more biomarkers within the exosomes as discussed herein (e.g. to create a genetic profile of the exosomes). The presence/absence of a certain biomarkers is used to indicate the presence/absence of the disease (e.g. cancer) in the subject as discussed herein. The process is performed periodically over time, and the results reviewed, to monitor the progression or regression of the disease, or to determine recurrence of the disease. A change in the amount of the biomarkers, particularly pBP1, indicates a change in the disease state in the subject, e.g., an increase in the level of pBP1 over time is indicative of disease progression. The period of time to elapse between sampling of exosomes from the subject, for performance of the isolation and analysis of the exosome, will depend upon the circumstances of the subject, and is to be determined by the skilled practitioner.

Selection of an individual from whom the exosomes are isolated is performed by the skilled practitioner based upon analysis of one or more of a variety of factors. Such factors for consideration are whether the subject has a family history of a specific disease (e.g. a cancer), has a genetic predisposition for such a disease, has an increased risk for such a disease due to family history, genetic predisposition, other disease or physical symptoms which indicate a predisposition, or environmental reasons. Environmental reasons include lifestyle, exposure to agents which cause or contribute to the disease such as in the air, land, water or diet. In addition, having previously had the disease, being currently diagnosed with the disease prior to therapy or after therapy, being currently treated for the disease (undergoing therapy), being in remission or recovery from the disease, are other reasons to select an individual for performing the methods.

The methods described herein are optionally performed with the additional step of selecting one or more biomarkers, e.g., pBP1 or pBP1 and another biomarker, e.g., the VEGF protein, for analysis. This selection can be based on any predispositions of the subject, or any previous exposures or diagnosis, or therapeutic treatments experienced or concurrently undergone by the subject.

The cancer diagnosed, monitored or otherwise profiled, can be any kind of cancer, but preferably includes, without limitation, cancers such as lung, ovarian, cervical, endometrial, breast, brain, colon and prostate cancers. Also included are gastrointestinal cancer, head and neck cancer, non-small cell lung cancer, cancer of the nervous system, kidney cancer, retina cancer, skin cancer, liver cancer, pancreatic cancer, genital-urinary cancer and bladder cancer, melanoma, and leukemia. In addition, the methods and compositions of the present invention are equally applicable to detection, diagnosis and prognosis of non-malignant tumors in an individual (e.g. neurofibromas, meningiomas and schwannomas).

In one embodiment, the cancer is, e.g., a breast cancer, prostate cancer, a leukemia, an ovarian cancer, or brain cancer.

The present invention further provides a kit for aiding in the detection or, or diagnosis of prognosis of, a cancer by detecting one or more biomarkers for a cancer in isolated exosomes. The kit comprises biomarker detecting reagents for detecting the one or more biomarkers in exosomes, and instructions for using the reagents to detect the biomarkers from isolated exosomes and aiding in the diagnosis or prognosis of the cancer. As used herein, the term “biomarker detecting reagents” refers to any substances, antibodies, receptors, chemicals, solutions used in reactions and processes that are capable of detecting, measuring, and examining the one or more biomarkers and their isoforms of interest. In one embodiments, the cancer is breast cancer and the biomarker is pBP-1, BP-1²⁵ and/or BP-1³⁷. In other preferred embodiments, the biomarker detecting reagents used herein comprise antibodies that specifically bind to BP-1, e.g., BP-1²⁵ or BPI³⁷, e.g. a polyclonal or monoclonal antibody that was raised against a BP-1 immunogenic fragment. For example, the kits may comprise a first monoclonal or polyclonal antibody raised against the peptide having the sequence SYPYTEPANPGSYLSCQQ SEQ ID NO:1. The kits of this invention may comprise a second monoclonal or polyclonal, wherein the second antibody binds to the first antibody. The antibodies may be antibodies coupled to a detectable substance, which facilitates their detection. Examples of detectable substances include various enzymes, prosthetic groups, fluorescent materials, luminescent materials, bioluminescent materials, radioactive materials, positron emitting metals using various positron emission tomographies, and nonradioactive paramagnetic metal ions. See, for example, U.S. Pat. No. 4,741,900 for metal ions which can be conjugated to antibodies for use in the present invention. Examples of suitable enzymes include horseradish peroxidase, alkaline phosphatase, beta-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin; and examples of suitable radioactive material include ¹²⁵I, ¹³¹I, ¹¹¹I or ⁹⁹TC.

The kit of the present invention further comprises an instruction for use in aiding in diagnosing, monitoring progression of, or in the prognosis of, breast cancer. In an embodiment, the instruction provides that an elevated pBP-1 level in isolated exosomes as compared to a negative control, e.g., the level of pBP1 in exosomes from subject known not to have breast cancer, indicates an association with breast cancer. In yet another embodiment, the instruction in the kit instructs the isolation of exosomes from a subject at various intervals, e.g., weekly, monthly, biannually, annually etc, and the measurement and comparison of pBP-1 levels in isolated exosomes using the detection reagents in the kit, and instructing that an increasing level of pBP-1, pBP-1³⁷ or pBp-1²⁵ in the isolated exosomes over time is associated with progression of a breast cancer, wherein a decreasing level of pBP-1, pBP-1³⁷ or pBp-1²⁵ in the isolated exosomes over time is associated with regression of a breast cancer.

The instructions in the kits of this invention may be present in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g., diskette, CD, etc., on which the information has been recorded. Yet another form that may be present is a website address which may be used via the internet to access the information at a removed site. Any convenient form may be present in the kits.

It should be understood that this invention is not limited to the particular methodologies, protocols and reagents, described herein and as such may vary. The terminology used herein is for the purpose of describing particular embodiments only, and is not intended to limit the scope of the present invention, which is defined solely by the claims.

### EXAMPLES

Isolation of Exosomes

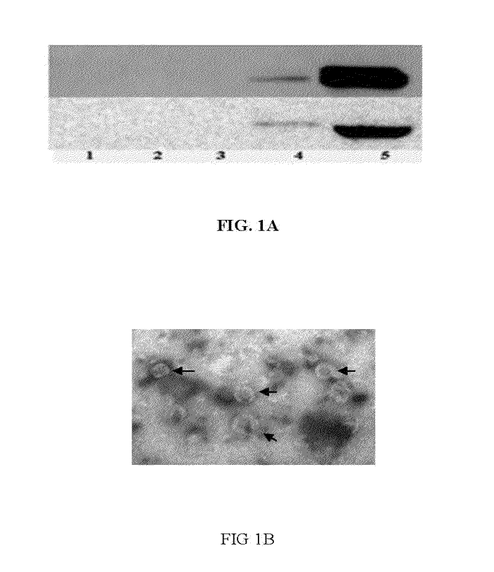

(a) Isolation of Exosomes. A method used herein for the isolation of exosomes from cell culture supernatants includes a series of centrifugations to remove large cellular debris, followed by high-speed ultracentrifugation to pellet exosomes (Thery et al. “Isolation and characterization of exosomes from cell culture supernatants and biological fluids” Curr. Protoc. Cell Biol. Chapter 3: Unit 3.22 (2006), incorporated herein by reference in its entirety). Briefly, conditioned media was centrifuged at 5000 rpm to eliminate cellular debris and subsequently concentrated in Centricon-Plus 70 (MWCO 10,000) filters (Millipore) at 2800 g for 20 minutes. The concentrated fraction was eluted by inverting the filter and centrifuging at 1000 g for 3 to 5 minutes (dependent on the volume remaining at the top of the filter). The concentrated conditioned media was then centrifuged in a Beckman TL-100 Ultracentrifuge. First, centrifugation at 10,000 g was performed twice, for 20 minutes each, to eliminate large cellular organelles and larger vesicles. The supernatant was then centrifuged at 100,000 g for 90 minutes and the pellet washed with a large volume of cold 1×PBS and recentrifuged at 100,000 g for another 90 minutes. All steps were carried out at 4° C. MDA-MB-231 breast cancer cells were examined for the presence of pBP1 in exosomes (FIG. 1). A small amount of supernatant was tested from each step, as well as the PBS from the washing step, and resolved on a Western blot alongside the exosome fraction to determine if any exosome lysis occurred as a result of the extraction process or if there is direct secretion of pBP1 into the media. Undamaged exosomes are evidenced by a lack of BP 1 protein and actin in the supernatant and the PBS samples. (Actin is found in exosomes as well as in cell extracts see Thery et al. “Exosomes: composition, biogenesis and function” Nat. Rev. Immunol. 8: 569-579 (2002) and Simpson et al. “Proteomic profiling of exosomes: current perspectives” Proteomics 8: 4083-4099 (2008)). pBP1 was observed in only the exosomal fraction cell extract (FIG. 1), but not in the supernatant or washes, consistent with pBP1 being secreted only in exosomes. This is the first transcription factor to our knowledge to be discovered in exosomes.

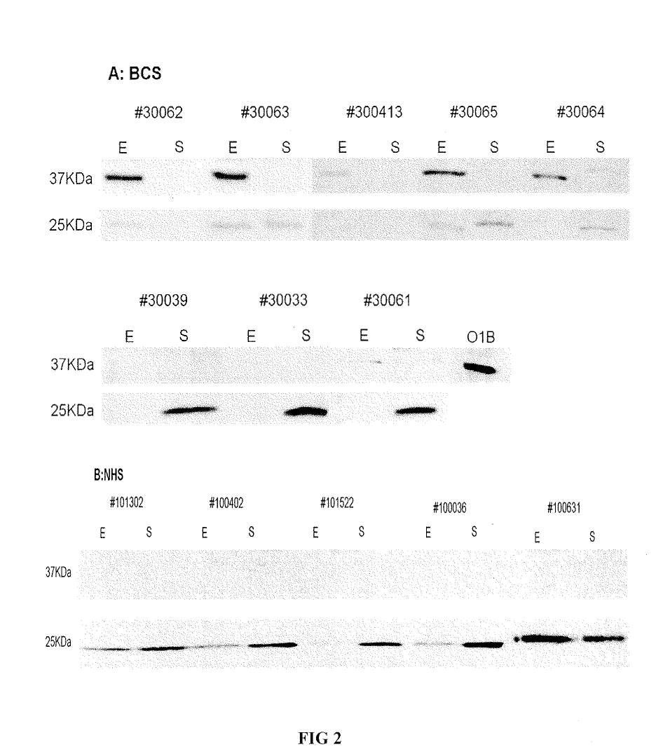

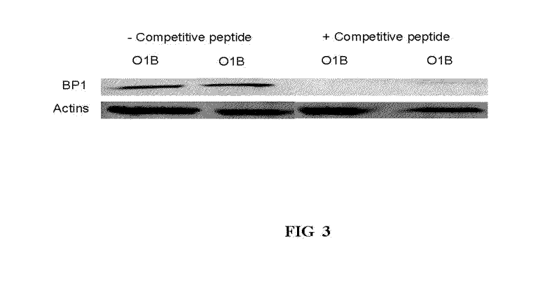

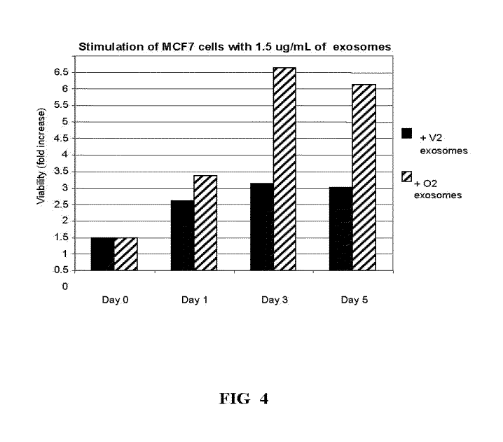

(b) pBP1 in serum exosomes. To determine if we could predict the ELISA results based on the level of pBP1 in exosomes as determined by Western blot analysis, we isolated exosomes from serum, then performed Western blotting on exosomes (Exo) and the supernatant (Sup) using an antibody specific for pBP1. FIG. 2 depicts the results obtained from samples of patients with metastatic breast cancer (BCS)(A), and from normal samples (NHS)(B). FIG. 2 depicts not only a larger band of approximately 37 kDa, but a second smaller band approximately 25 kDa band, referred to hereinafter pBP-1²⁵, not previously observed. The larger band, which has been observed in cell extracts from breast cancer cell lines, binds specifically to the anti-BP1 antibody since it is competed by the peptide used to immunize rabbits to produce the anti-BP1 antibody (FIG. 3). The pBP-1²⁵ band was not observed in the cell extracts, but it is the predicted size for unmodified BP1 protein (Chase et al. “BP1, a homeodomain-containing isoform of DLX4, represses the beta-globin gene” Mol. Cell. Biol. 22: 2505-2514 (2002)).

Surprisingly, the pBP-1²⁵ band is the only band detected in NHS; it is in the supernatant fraction (Sup), without wishing to be bound by theory raising the possibility that it is secreted independently of exosomes. In BCS, when pBP1³⁷ was present it was in exosome fraction (Exo), not the Sup; three BCS samples (30039, 30033 and 30061) exhibit only pBP1²⁵ and the pBP1²⁵ was only detected in the Sup, similar to NHS. These data indicate that pBP1²⁵ is associated with NHS and, without wishing to be bound by theory when found in breast cancer, may perhaps be associated with better prognosis.

Conclusions. We have demonstrated that pBP1 is secreted in exosomes found in both normal serum and serum from women with metastatic breast cancer. There are two protein bands recognized by our anti-BP1 antibody, pBP1²⁵ and pBP1³⁷. The 25 kDa band is predominantly found in the supernatant of NHS; NHS lacks pBP1³⁷. pBP1³⁷ is only present in exosomes of BCS. pBP1³⁷ is specifically recognized by our antibody.

The results disclosed herein indicate that pBP1³⁷ is present at a higher level in exosomes from serum from breast cancer patients than serum from normal individuals and that high pBP1³⁷ levels are likely associated with a poorer outcome.

Without wishing to be bound by theory, higher levels of pBP1 in BCS than NHS, may predict decreased overall survival. Detection of pBP1 in serum, particularly in the exosomes, of women with metastatic breast cancer suggests pBP1 may be a useful tool for classification of various stages of breast cancer with respect to their progression and outcome. Measurement of serum pBP1, particularly in the exosomes, could also be useful in monitoring therapy and early detection.

We claim:

1. A method for aiding in the diagnosis of, or monitoring of progression or regression of, a breast cancer in a subject, comprising
(a) obtaining an exosome-containing bodily fluid sample from a subject in need thereto,
(b) isolating exosomes from the bodily fluid sample,
(c) detecting the one or more biomarkers in the exosomes of (b),
wherein the one or more biomarkers comprise pBP-1 and wherein detection of BP-1 in exosomes is associated with breast cancer in the subject thereby aiding in the diagnosis of, or monitoring of progression or regression of, breast cancer.

2. The method for claim 1 further comprising
(d) comparing the amount of the one or more biomarkers in the exosomes determined in (c) to one or more positive or negative control amounts,
wherein a amount of pBP1 in the exosomes of the subject that is (i) significantly higher than the amount of pBP1 in the negative control or (ii) at least the amount of pBP1 of the positive control, aids in the diagnosis of, or monitoring the progression or regression of, a breast cancer in the subject.

3. The method of claim 1 wherein the BP-1 is BP-1²⁵ or BP-1³⁷ or both.

4. The method of claim 1, wherein the bodily fluid sample comprises milk, tears, urine, saliva, sputum, blood, serum, plasma, ascites, cyst fluid, pleural fluid, cerebral spinal fluid, or combinations thereof.

5. The method of claim 1 wherein the bodily fluid is a blood or serum sample.

6. The method of claim 1, wherein isolating the exosomes comprises using differential ultracentrifugation, size exclusion chromatography, and ultrafiltration to isolate the exosomes.

7. The method of claim 1, wherein the exosomes are selectively isolated by immunoabsorbent capture using one or more cancer specific antigen binding antibody, wherein the cancer specific antigen comprises, CD70, carcinoembryonic antigen (CEA), EGFR, EGFRvIII and other variants, Fas ligand, TRAIL, tranferrin receptor, p38.5, p97, HSP72, CD63, or HER2.

8. The method of claim 1, wherein detecting the one or more biomarkers comprises capturing the one or more biomarker with one or more antibodies or receptors that each selectively bind to pBP-1.

9. The method of claim 1, comprising collecting exosome containing bodily fluid samples at various time intervals and subjecting the samples to steps (a)-(c) and comparing the levels of the one or more biomarkers in the various samples, wherein an increase or decrease in the level of the one or more biomarkers over time is indicative of the progression or regression of breast cancer in the subject.

10. The method of claim 1 wherein the biomarkers further comprises vascular endothelial growth factor (VEGF).

11. The method of claim 1 wherein the breast cancer is ductal hyperplasia (IDH), ductal carcinoma in situ (DCIS), invasive ductal carcinoma (IDC) or inflammatory breast cancer (IBC).

12. The method of claim 1, wherein the one or more biomarkers are detected by ELISA or by Western blot.

13. The method of claim 1 wherein the pBP1 is pBP1³⁷ or pBP1²⁵ or both.

14. A kit for aiding in the diagnosis of, or monitoring the progression or regression of a breast cancer comprising
(a) at least one of
(i) antibodies that bind specifically to BP-1, which antibodies are affixed to a solid support,
(ii) labeled antibodies that bind specifically to BP-1
(b) instructions for (i) isolating exosomes from a bodily fluid, (ii) then isolating polypeptides from the isolated exosomes of (b)(i), and (iii) then detecting BP-1 among the polypeptides of (b)(ii) by using antibodies of (a)(i) or (a)(ii) or combinations of (a)(i) and (a)(ii)
wherein said instructions disclose a negative control level of BP-1 in exosomes of NHS or a positive control level of BP-1 in exosomes from a BCS bodily fluid.

15. The kit of claim 14, wherein the antibodies specifically bind a peptide comprising the sequence SYPYTEPANPGSYLSCQQ (SEQ ID NO:1), or BP-1³⁷ or BP-1²⁵.

16. The kit of claim 14, wherein the antibodies are fluorescently or radioactively labeled.


SEQUENCE LISTING (1 sequence)

SEQ ID NO: 1 - protein, 18 aa, homo sapiens
SYPYTEPANPGSYLSCQQ